(19) United States
(12) Patent Application Publication
(10) Pub. No.: US 2017/0100086 A1
(43) Pub. Date: Apr. 13, 2017

(54) LUMEN PASSABILITY CHECKING DEVICE

(71) Applicant: OLYMPUS CORPORATION, Tokyo (JP)

(72) Inventors: Kei TAKASUGI, Tokyo (JP); Akio UCHIYAMA, Yokohama-shi (JP)

(73) Assignee: OLYMPUS CORPORATION, Tokyo (JP)

(21) Appl. No.: 15/381,416

(22) Filed: Dec. 16, 2016

Related U.S. Application Data
(63) Continuation of application No. PCT/JP2015/073345, filed on Aug. 20, 2015.

(30) Foreign Application Priority Data
Nov. 6, 2014 (JP) 2014-226232

(51) Int. Cl. A61B 6/00; A61M 31/00; A61B 5/00; A61B 1/04; A61B 5/107

(52) U.S. Cl. CPC A61B 6/481; A61B 1/041; A61B 5/1076; A61B 5/4255; A61M 31/005

(57) ABSTRACT

A lumen passability checking device checks whether a capsule medical device configured to be introduced into a gastrointestinal tract of a subject is allowed to pass through the gastrointestinal tract. The device includes: a main body dissolvable by a substance in the gastrointestinal tract; a first layer coating the main body, having an outer diameter substantially equivalent to an outer diameter in a minor axis of the capsule medical device, and being dissolvable by the substance in the gastrointestinal tract; and a second layer coating the first layer and being dissolvable by the substance in the gastrointestinal tract. The first and second layers contain different discriminating materials. The first layer has at least two regions extending along a circumference of a cross section of the first layer, the cross section intersecting with a long axis of the device, a part of each region being thinner than other parts.

### CROSS REFERENCES TO RELATED APPLICATIONS

This application is a continuation of PCT international application Ser. No. PCT/JP2015/073345 filed on Aug. 20, 2015 which designates the United States, incorporated herein by reference, and which claims the benefit of priority from Japanese Patent Application No. 2014-226232, filed on Nov. 6, 2014, incorporated herein by reference.

### BACKGROUND

1. Technical Field

The disclosure relates to a device for checking lumen passability of a capsule medical device configured to be introduced into a body of a subject.

2. Related Art

In recent years, in a field of endoscope, a capsule endoscope having both an imaging function and a wireless communication function is emerging. For observation (examination) of internal portions of organs, the capsule endoscope is swallowed from the mouth of the subject such as a patient and thereafter, for a period until being discharged naturally from the subject, moves inside the organs such as the stomach and the small intestine with peristaltic action, or the like, and sequentially captures an image inside the organs of the patient (hereinafter, referred to as an in-vivo image in some cases) with an interval of 0.5 second, for example. The capsule endoscope sequentially transmits wirelessly the in-vivo image captured in this manner, to a receiving device carried by the subject.

The in-vivo image wirelessly transmitted by the capsule endoscope is sequentially received by the receiving device and sequentially stored in a storage medium of the receiving device. An image display device obtains an in-vivo image group of the subject via the storage medium of the receiving device and displays the in-vivo image group of the subject, on a display. A doctor, a nurse, or the like, observes the in-vivo image displayed on the image display device and diagnoses the subject.

If a stenosis portion exists inside an organ (inside the lumen) of the subject, this type of capsule endoscope might stagnate at the stenosis portion. To handle this, the doctor, the nurse, or the like, needs to check lumen passability of the capsule endoscope ingested by the subject, before performing capsule endoscope examination on the subject. For this purpose, a lumen passability checking device is ingested by the subject. The lumen passability checking device has an outer diameter substantially equivalent to the outer diameter of the capsule endoscope for which lumen passability is to be checked. On the basis of a result of whether the lumen passability checking device has been discharged from the body, or whether the device has reached a desired organ positioned beyond the organ with the stenosis portion, whether the capsule endoscope used for examination can pass through the lumen is determined. As this lumen passability checking device, for example, a lumen passability checking device is proposed in which the surface of a capsule main body, made of a material that is easily dissolvable inside the living body, is substantially uniformly coated with a low-solubility material so as to maintain a certain level of durability inside the body of the subject (for example, refer to JP Patent 4307995 B).

### SUMMARY

In some embodiments, provided is a lumen passability checking device for checking whether a capsule medical device configured to be introduced into a gastrointestinal tract of a subject is allowed to pass through the gastrointestinal tract. The device includes: a main body made of a material dissolvable by a substance existing in the gastrointestinal tract; a first dissolving layer coating a surface of the main body, having an outer diameter substantially equivalent to an outer diameter in a minor axis of the capsule medical device, and being made of a material dissolvable by the substance existing in the gastrointestinal tract; and a second dissolving layer coating a surface of the first dissolving layer and being made of a material dissolvable by the substance existing in the gastrointestinal tract. The first and second dissolving layers contain different discriminating materials. The first dissolving layer has at least two regions extending along a circumference of a cross section of the first dissolving layer, the cross section intersecting with a long axis of the lumen passability checking device, at least a part of each of the two regions being thinner than other parts.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a perspective view of a lumen passability checking device according to a first embodiment;

FIG. 2 is a cross-sectional view of the lumen passability checking device illustrated in FIG. 1, cut along a plane that includes a long axis of the lumen passability checking device and that is parallel to the long axis;

FIG. 3 is a schematic view illustrating a state where the lumen passability checking device illustrated in FIG. 1 is orally ingested;

FIGS. 4A to 4C are diagrams illustrating a state in which the lumen passability checking device illustrated in FIG. 2 dissolves in the intestinal tract of the small intestine;

FIG. 5 is a cross-sectional view of the lumen passability checking device according to a modification example of the first embodiment, cut along a plane that includes a long axis of the lumen passability checking device and that is parallel to the long axis;

FIG. 6 is a side view of a main body of a lumen passability checking device according to a second embodiment;

FIG. 7 is a cross-sectional view of the lumen passability checking device according to the second embodiment, cut along a plane that includes a long axis of the lumen passability checking device and that is parallel to the long axis;

FIGS. 8A to 8C are diagrams illustrating a state in which the lumen passability checking device illustrated in FIG. 7 dissolves in the intestinal tract of the small intestine;

FIG. 9 is a side view of a main body of a lumen passability checking device according to a first modification example of the second embodiment;

FIG. 10 is a cross-sectional view of the lumen passability checking device according to a first modification example of the second embodiment, cut along a plane that includes a long axis of the lumen passability checking device and that is parallel to the long axis;

FIG. 11 is a diagram illustrating a state in which the lumen passability checking device illustrated in FIG. 10 dissolves in the intestinal tract of the small intestine;

FIG. 12 is a side view of a main body of a lumen passability checking device according to a second modification example of the second embodiment;

FIG. 13 is a diagram illustrating a state in which the lumen passability checking device illustrated in FIG. 12 dissolves in the intestinal tract of the small intestine;

FIG. 14 is a side view of a main body of a lumen passability checking device according to a third modification example of the second embodiment;

FIG. 15 is a cross-sectional view of the lumen passability checking device according to the third modification example of the second embodiment, cut along a plane that is orthogonal to the long axis of the lumen passability checking device and that passes through a protrusion of the main body;

FIG. 16 is a side view of a lumen passability checking device according to a third embodiment;

FIG. 17 is a cross-sectional view of the lumen passability checking device according to the third embodiment, cut along a plane that includes a long axis of the lumen passability checking device and that is parallel to the long axis;

FIG. 18 is a side view of a lumen passability checking device according to a first modification example of the third embodiment;

FIG. 19 is a side view of a lumen passability checking device according to a second modification example of the third embodiment;

FIG. 20 is a side view of a lumen passability checking device according to a third modification example of the third embodiment;

FIG. 21 is a cross-sectional view of FIG. 20, taken along line A-A;

FIG. 22 is a cross-sectional view of a lumen passability checking device according to a fourth embodiment, cut along a plane that includes a long axis of the lumen passability checking device and that is parallel to the long axis;

FIG. 23 is a diagram illustrating an X-ray photographic image of the lumen passability checking device in a state where a first coating layer illustrated in FIG. 22 remains on the whole surface of the device;

FIG. 24 is a diagram illustrating an X-ray photographic image of the lumen passability checking device in a state where the first coating layer has dissolved and a second coating layer is exposed, illustrated in FIG. 22;

FIG. 25 is a cross-sectional view of another lumen passability checking device according to a fourth embodiment, cut along a plane that includes a long axis of the lumen passability checking device and that is parallel to the long axis;

FIG. 26 is a cross-sectional view of another lumen passability checking device according to the fourth embodiment, cut along a plane that includes a long axis of the lumen passability checking device and that is parallel to the long axis; and

FIG. 27 is a cross-sectional view of a lumen passability checking device according to a fifth embodiment, cut along a plane that includes a long axis of the lumen passability checking device and that is parallel to the long axis.

### DETAILED DESCRIPTION

Hereinafter, preferred embodiments of a lumen passability checking device according to the present invention will be described in detail with reference to the drawings. Note that hereinafter, a capsule-shaped device will be described as an exemplary lumen passability checking device according to the present invention. The present invention is not intended to be limited by these embodiments. The same reference signs are used to designate the same elements throughout the drawings.

### First Embodiment

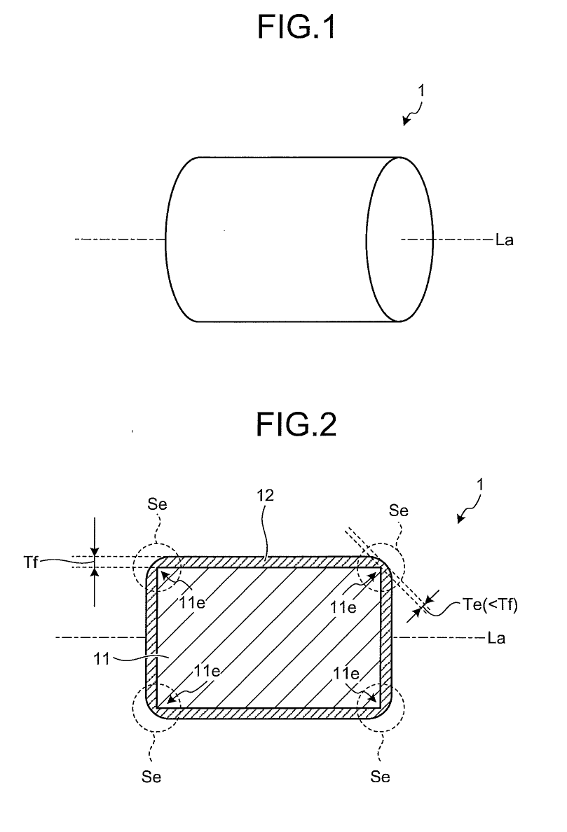

FIG. 1 is a perspective view of a lumen passability checking device according to a first embodiment. As illustrated in FIG. 1, a lumen passability checking device 1 according to the first embodiment is a device for checking whether a capsule medical device introduced into the gastrointestinal tract of a subject can pass through an organ as a passability check target. Therefore, the lumen passability checking device 1 has an external shape similar to that of the capsule medical device to be introduced into the gastrointestinal tract of the subject so that the lumen passability checking device 1 can be introduced into the gastrointestinal tract of the subject. The lumen passability checking device 1 has an outer diameter substantially equivalent to an outer diameter in a minor axis of the capsule medical device to be introduced. The lumen passability checking device 1 is substantially columnar, for example. Even when external pressure is applied with peristaltic action of the organ, the lumen passability checking device 1 maintains the outer diameter substantially equivalent to that of the capsule medical device to be introduced, and further maintains the external shape similar to that of the capsule medical device.

FIG. 2 is a cross-sectional view of the lumen passability checking device 1 illustrated in FIG. 1, cut along a plane that includes a long axis La of the lumen passability checking device 1 and that is parallel to the long axis La. As illustrated in FIG. 2, the lumen passability checking device 1 includes a columnar main body 11 and a coating layer (dissolving layer) 12. The coating layer (dissolving layer) 12 is configured to coat (cover) an outer surface of the main body 11.

The main body 11 is made of a material dissolvable by a substance existing in the gastrointestinal tract, namely, the organ as a passability check target of the capsule medical device. The main body 11 is made of a material that dissolves more easily than the coating layer 12. The dissolution rate of the main body 11 is higher than the dissolution rate of the coating layer 12 to be described below. Since the main body 11 is columnar, a circumference portion 11e (refer to FIG. 2) as a boundary between a bottom surface and a side surface extends along each of outer peripheries of the two bottom surfaces of the cylinder. In examining passability through the intestinal tract of the small intestine, the target organ to be reached by the lumen passability checking device 1 is the large intestine. Accordingly, the main body 11 is made of an enteric material dissolvable in a short time by the liquid (intestinal fluid or digestive fluid) existing in the intestinal tract of the small intestine and the large intestine.

The coating layer 12 has an outer diameter substantially equivalent to the outer diameter in a minor axis of the capsule medical device to be introduced, and is made of a material dissolvable by a substance existing in the gastrointestinal tract as a passability check target. The coating layer 12 has a substantially cylindrical shape with closed ends so as to contain the columnar main body 11. The coating layer 12 includes at least a material that dissolves by the liquid (intestinal fluid or digestive fluid) existing in the gastrointestinal tract as the passability check target for the capsule medical device. In examining passability through the intestinal tract of the small intestine, the target organ to be reached by the lumen passability checking device 1 is the large intestine. Accordingly, the coating layer 12 is made of an enteric material that dissolves with time by the liquid existing in the intestinal tract of the short intestine and the large intestine. For example, the coating layer 12 is made of a general enteric material such as gelatin that gradually dissolves in any of the small intestine and the large intestine. Note that the enteric material dissolves by the digestive fluid, or the like, inside the intestines. The coating layer 12 is formed by spraying or depositing an enteric material, for example, to the main body 11.

The coating layer 12 is configured such that a thickness Te of a portion coating the circumference portion 11e of the main body 11, represented in a region Se, is thinner than a thickness Tf of a portion coating the side surface and the bottom surface of the main body 11. In other words, the coating layer 12 is configured such that the thickness Te coating the circumference portion 11e of the main body 11 extending along the outer periphery of the bottom surface around the long axis La of the lumen passability checking device 1 is thinner than the thickness Tf of other portions.

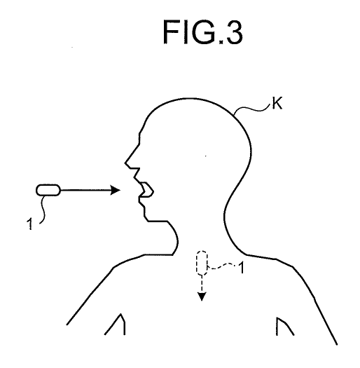

As illustrated in FIG. 3, for example, the above-configured lumen passability checking device 1 is orally ingested by a subject K before the capsule medical device is introduced into the body (specifically, before it is orally ingested). The lumen passability checking device 1 orally ingested by the subject K moves ahead inside the lumen of the subject K with peristaltic action, or the like. In a case where there is no stenosis portion inside the lumen of the subject K, the lumen passability checking device 1 reaches the large intestine as a reaching target, or discharged from the body.

In another a case where there is a stenosis portion inside the lumen of the subject K, the lumen passability checking device 1 stagnates at the stenosis portion. Note that the lumen passability checking device 1 maintains its shape by the coating layer 12 until it reaches the small intestine. The lumen passability checking device 1 dissolves with time when it stagnates at the stenosis portion of the small intestine.

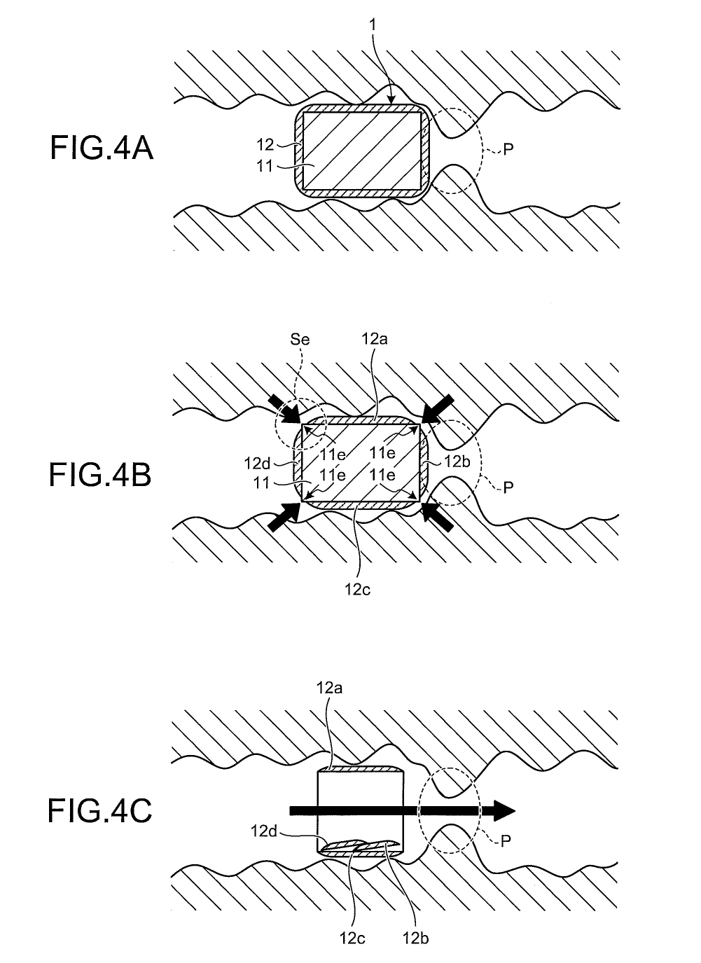

FIGS. 4A to 4C are diagrams illustrating a state in which the lumen passability checking device 1 dissolves in the intestinal tract of the small intestine. As illustrated in FIG. 4A, when the lumen passability checking device 1 stagnates at a stenosis portion P of the small intestine, the coating layer 12 comes to a state, as illustrated in FIG. 4B, in which a thin portion, in thickness, coating the two circumference portions 11e inside the region Se dissolves prior to the other portions. As a result, the coating layer 12 is divided into portions 12b and 12d coating the bottom surfaces of the main body 11, and cylindrical portions 12a and 12c coating the side surface of the main body 11, and then, the surface of the circumference portion 11e of the main body 11 is exposed from a gap on each of the portions 12a to 12d. As illustrated with the arrow in FIG. 4B, the liquids existing inside the small intestine enter from the exposed circumference portion 11e of the main body 11, and the whole main body 11 dissolves as illustrated in FIG. 4C. As a result, among the divided coating layer 12, the portions 12b and 12d coating the bottom surfaces of the main body 11 fall down inside the cylindrical portions 12a and 12c coating the side surface of the main body 11, and the coating layer 12 remains in a state where at least both ends of the cylindrical shape communicates with each other. With this state, even in a case where the lumen passability checking device 1 stagnates at the stenosis portion P to cause the coating layer 12 to remain there, intestinal fluid, or the like, can pass through the internal portion of the coating layer 12, as illustrated with the arrow in FIG. 4C.

In this manner, according to the first embodiment, by forming the portion to be divided to be thinner than the other portions, on the coating layer 12, it is possible to divide the coating layer 12 at a desired position. On the lumen passability checking device 1, the coating layer 12 is configured to form the thickness of a pattern that extends along the cross section intersecting with the long axis La of the lumen passability checking device 1, for example, the thickness of the portion coating the circumference portion 11e of the main body 11, to be thinner than the thickness of the other portions. With this configuration, the bottom surface portions of the coating layer 12 having a cylindrical shape are allowed to be divided from the cylindrical portion. As a result, even in a case where the lumen passability checking device 1 stagnates at the stenosis portion P to cause the coating layer 12 to remain inside the intestinal tract, intestinal fluid, or the like, can pass through the internal portion of the cylindrical-shaped coating layer 12 with both ends thereof communicating with each other, making it possible to reduce the effects on the living body.

### Modification Example of First Embodiment

FIG. 5 is a cross-sectional view of a lumen passability checking device according to a modification example of the first embodiment, cut along a plane that includes a long axis of the lumen passability checking device and that is parallel to the long axis. As illustrated with a lumen passability checking device 1A in FIG. 5, it is also possible to separately provide cylindrical portions 121 and 123 coating the side surfaces of the main body 11, and bottom surface portions 122 and 124 coating the bottom surfaces of the main body 11, and to join the cylindrical portions 121 and 123 with the bottom surface portions 122 and 124 using adhesive 13, thereby generating a coating layer. In this case, the coating layer dissolves from a boundary portion between the cylindrical portions 121 and 123 and the bottom surface portions 122 and 124, and thus, the cylindrical portions 121 and 123 are divided from the bottom surface portions 122 and 124.

### Second Embodiment

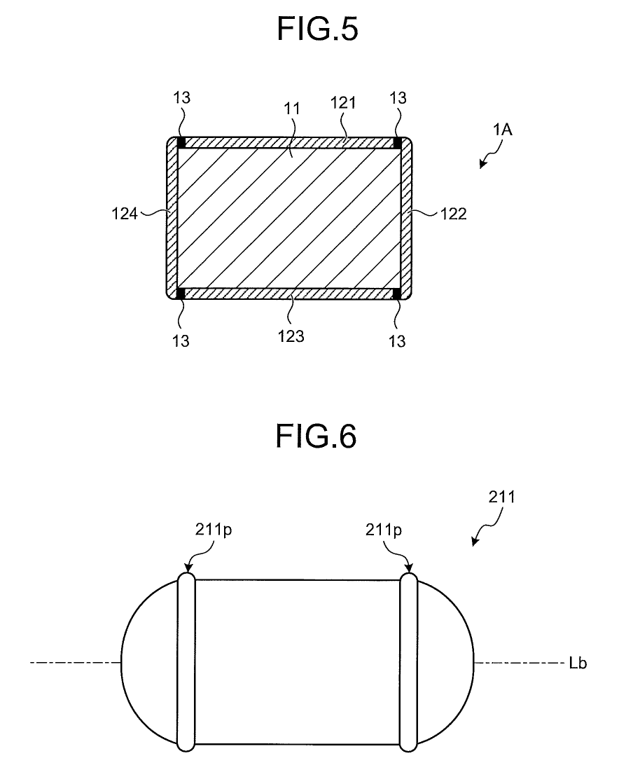

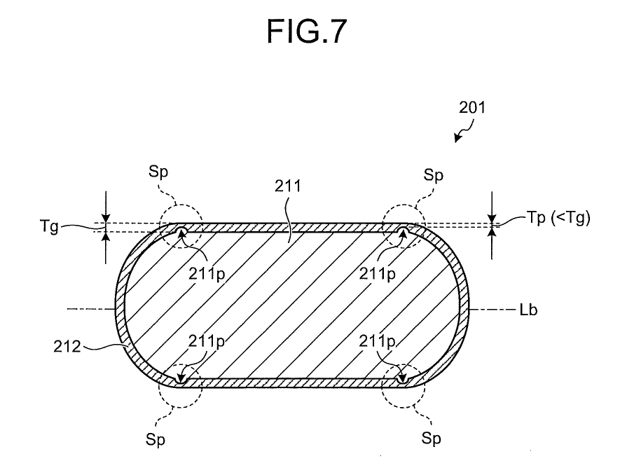

Next, a second embodiment will be described. FIG. 6 is a side view of a main body of a lumen passability checking device according to the second embodiment. FIG. 7 is a cross-sectional view of a lumen passability checking device 201 according to the second embodiment, cut along a plane that includes a long axis Lb of the lumen passability checking device 201 and that is parallel to the long axis Lb.

As illustrated in FIG. 6, in the lumen passability checking device 201 according to the second embodiment, two protrusions 211p are formed on outer surfaces on the side of the end portion of a capsule-shaped main body 211. The protrusion 211p extends along a cross section of the main body 211 that is orthogonal to the long axis Lb of the lumen passability checking device 201. Therefore, as illustrated in FIG. 7, a coating layer 212 coating the outer surface of the main body 211 is configured such that a thickness Tp of a portion coating the protrusion 211p of the main body 211 inside the region Sp is thinner than a thickness Tg of a portion coating the other portions of the main body 211.

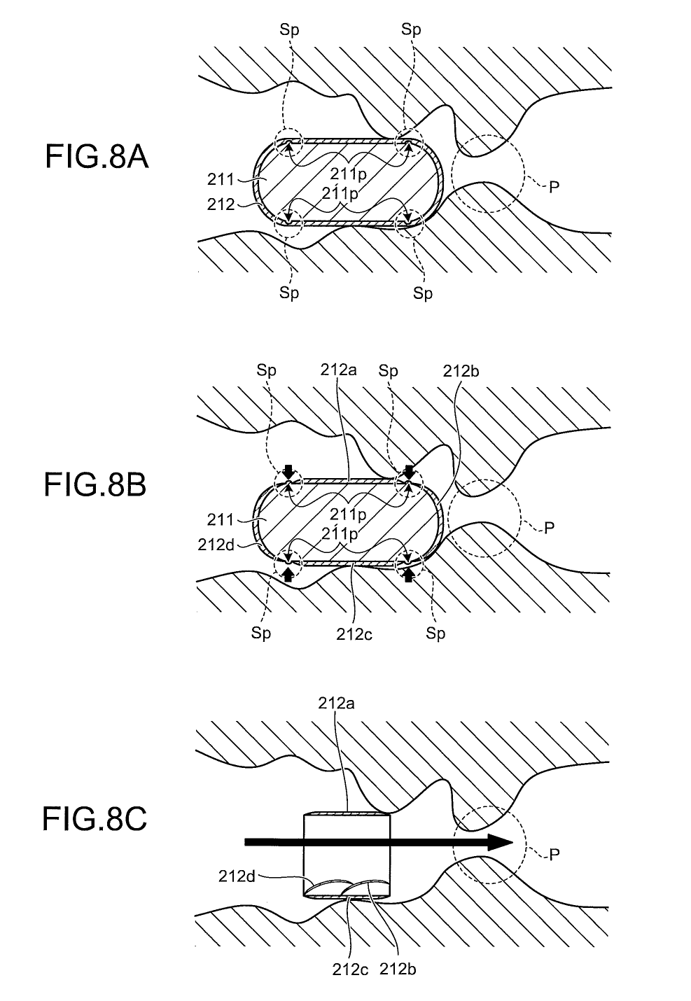

FIGS. 8A to 8C are diagrams illustrating a state in which the lumen passability checking device 201 dissolves in the intestinal tract of the small intestine. As illustrated in FIG. 8A, when the lumen passability checking device 201 stagnates at a stenosis portion P of the small intestine, the coating layer 212 comes to a state, as illustrated in FIG. 8B, in which a thin portion, in thickness, coating the protrusion 211p inside the region Sp dissolves prior to the other portions. Accordingly, the coating layer 212 is configured such that opening extending along a cross section orthogonal to the long axis Lb of the lumen passability checking device 201 opens corresponding to the forming position of the protrusion 211p. As a result, the coating layer 212 is divided into cylindrical portions 212a and 212c coating a barrel part of the main body 211 and portions 212b and 212d coating ends of the main body 211. Accordingly, the surface of the protrusion 211p of the main body 211 is exposed from the gap between each of the portions 212a to 212d. Subsequently, the liquids existing inside the small intestine enters from the exposed protrusion 211p of the main body 211, as illustrated in FIG. 8B. When the whole of the main body 211 has dissolved as illustrated in FIG. 8C, each of the divided portions of the coating layer 212 scatters, and at least both ends of the cylindrical portions 212a and 212c come to a state of communicating with each other, on the coating layer 212. In this state, intestinal fluid, or the like, can pass through the internal portion of the coating layer 212, as illustrated with the arrow.

In this manner, in the second embodiment, the protrusion 211p that extends along the circumference of the cross section orthogonal to the long axis Lb of the lumen passability checking device 201 is provided on the surface of the main body 211, whereby the coating layer 212 on the protrusion 211p is thinner than the other portions of the coating layer 212. In other words, the coating layer 212 is configured such that the thickness of the cross section of the pattern on the protrusion 211p that extends along the circumference of two cross sections orthogonal to the long axis Lb of the lumen passability checking device 201 is formed to be thinner than the thickness of the portion coating portions other than the protrusion 211p. As a result, the lumen passability checking device 201 allows pattern sections of the coating layer 212 on the protrusion 211p to dissolve fast, and allows the remaining coating layer 212 to be divided into a cylindrical portion having at least both ends communicating with each other, and the other portions, making it possible to achieve effects similar to the effects of the first embodiment. In the second embodiment, the protrusion 211p not only extends along the circumference of the cross section orthogonal to the long axis Lb of the lumen passability checking device 201, but also may extend along a curved surface intersecting with the long axis Lb.

### First Modification Example of Second Embodiment

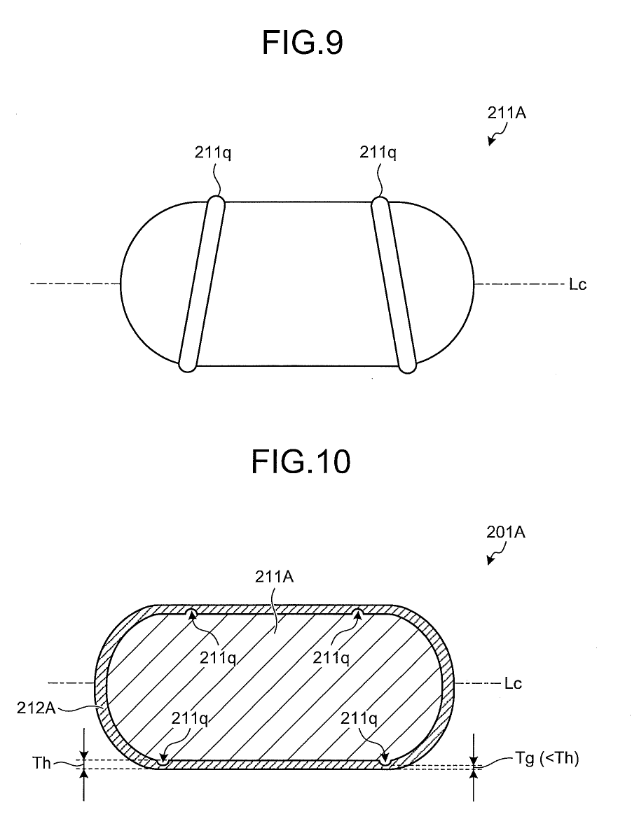

FIG. 9 is a side view of a main body of a lumen passability checking device according to a first modification example of the second embodiment. FIG. 10 is a cross-sectional view of a lumen passability checking device 201A according to the first modification example of the second embodiment, cut along a plane that includes a long axis Lc of the lumen passability checking device 201A and that is parallel to the long axis Lc.

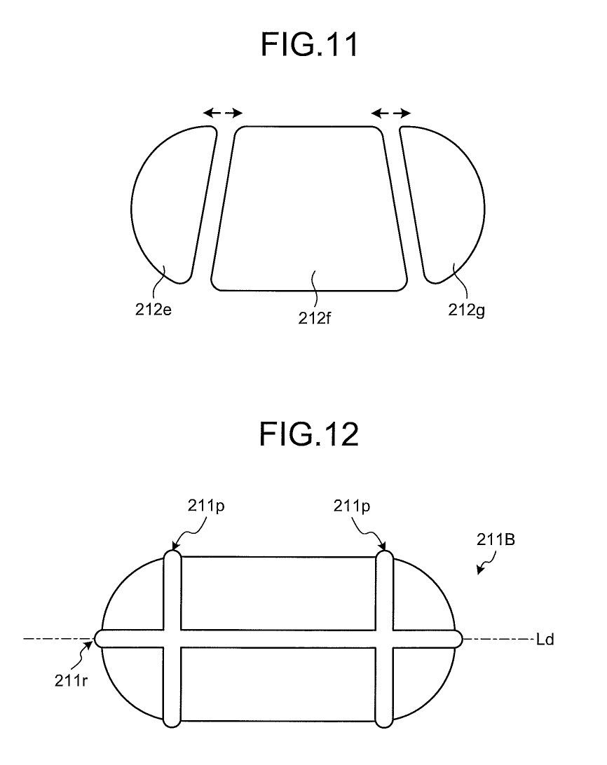

As illustrated in FIGS. 9 and 10, the lumen passability checking device 201A according to the first modification example of the second embodiment is configured such that two protrusions 211q that extend along a cross section intersecting with the long axis Lc of the lumen passability checking device 201A are formed on a main body 211A. Also in this case, a coating layer 212A coating the outer surface of the main body 211A is configured such that a thickness Tg of a portion coating the protrusion 211q of the main body 211A is thinner than a thickness Th of a portion coating the other portions of the main body 211A. Therefore, in a case where the lumen passability checking device 201A stagnates at a stenosis portion of the small intestine, the coating layer 212A comes to a state where the thin portion, in thickness, coating the protrusion 211q dissolves prior to the other portions, and the coating layer 212A is divided into end portions 212e and 212g, and a cylindrical portion 212f, as illustrated in FIG. 11. With this configuration, the coating layer 212A comes to a state where at least the cylindrical portion 212f has its both ends thereof communicating with each other, allowing intestinal fluid, or the like, to pass through the internal portion of the coating layer 212A. In order to divide the coating layer remaining inside the body, into the end portions and the cylindrical portion, there is no need to use a cross section orthogonal to the long axis of the lumen passability checking device. Alternatively, it would be sufficient to form a protrusion to extend along the circumference of a cross section intersecting with the long axis of the lumen passability checking device, as the protrusion 211q formed on the main body 211A. Further alternatively, the protrusion 211q may be provided to extend along the circumference of a curved surface intersecting with the long axis Lc of the lumen passability checking device 201A.

### Second Modification Example of Second Embodiment

FIG. 12 is a side view of a main body of a lumen passability checking device according to a second modification example of the second embodiment.

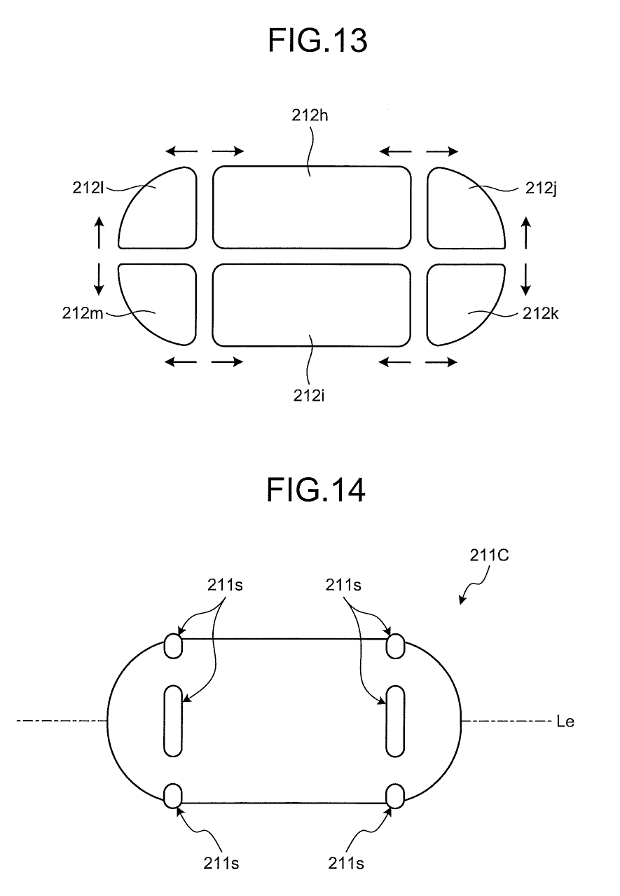

As illustrated in FIG. 12, a lumen passability checking device according to the second modification example of the second embodiment is configured such that, in addition to the two protrusions 211p, a protrusion 211r is provided to extend along the circumference of a cross section of a main body 211B, cut along a plane including a long axis Ld, on the main body 211B. Also in this case, a coating layer coating the outer surface of the main body 211B is configured such that a portion coating the protrusions 211p and 211r of the main body 211B is thinner than a portion coating the other portions of the main body 211B. Subsequently, in a case where the lumen passability checking device according to the second modification example of the second embodiment stagnates at a stenosis portion of the small intestine, the coating layer comes to a state where the thin portions, in thickness, coating the protrusions 211p and 211r dissolve prior to the other portions, and the coating layer is divided into portions 212j to 212m generated by dividing end portions at the position of the protrusion 211r, and portions 212h and 212i generated by dividing the cylindrical portion at the position of the protrusion 211r, as illustrated in FIG. 13.

In this manner, the coating layer of the lumen passability checking device can be divided further finely along a plane passing through the long axis, by not only forming the protrusion provided to extend along the circumference of the cross section intersecting with the long axis of the lumen passability checking device, but also forming a protrusion provided to extend along the circumference of the cross section of the main body, cut along the plane including the long axis of the lumen passability checking device.

### Third Modification Example of Second Embodiment

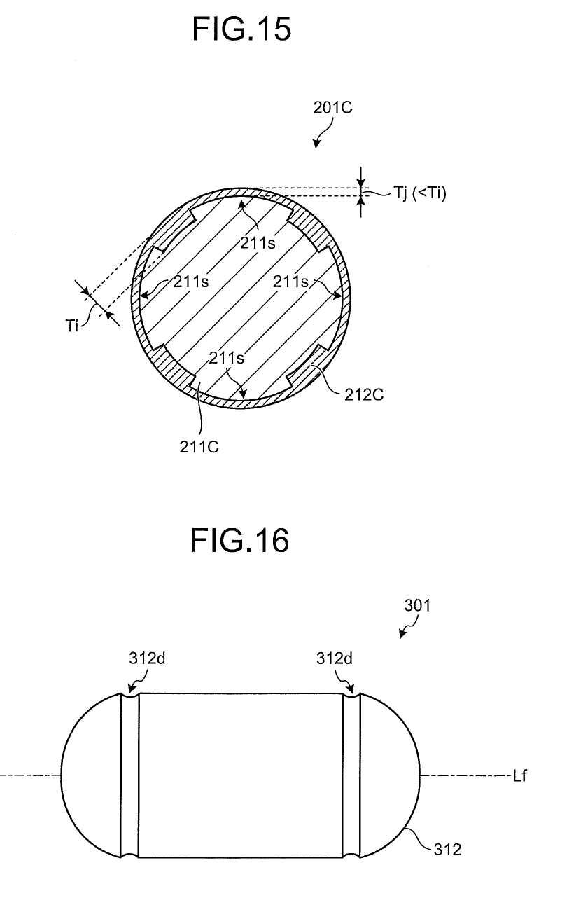

FIG. 14 is a side view of a main body of a lumen passability checking device according to a third modification example of the second embodiment. FIG. 15 is a cross-sectional view of a lumen passability checking device 201C according to the third modification example of the second embodiment, cut along a plane that is orthogonal to a long axis Le of the lumen passability checking device 201C and that passes through a protrusion of the main body.

As illustrated in FIGS. 14 and 15, the lumen passability checking device 201C according to the third modification example of the second embodiment is configured such that two sets of a plurality of protrusions 211s are provided on a main body 211C, along the circumference of the cross section of the main body 211C, intersecting with a long axis Le of the lumen passability checking device 201C. In other words, as illustrated in FIG. 15, on the main body 211C of the lumen passability checking device 201C, two sets of the plurality of protrusions 211s are partially formed along the circumference of the cross section intersecting with the long axis Le of the lumen passability checking device 201C, instead of the protrusion extending along entire portions of the circumference of the cross section intersecting with the long axis Le of the lumen passability checking device 201C. Also in this case, a coating layer 212C coating the outer surface of the main body 211C is configured such that a thickness Tj of a portion coating the protrusion 211s of the main body 211C is thinner than a thickness Ti of a portion coating the other portions of the main body 211C.

If the lumen passability checking device 201C according to the third modification example of the second embodiment stagnates at a stenosis portion of the small intestine, the coating layer 212C comes to a state where the thin portions, in thickness, coating the protrusion 211s dissolve prior to the other portions, and perforation-like holes are generated to open along the positions of the protrusions 211s. With this arrangement, the coating layer is easily divided into both end portions and the cylindrical portion, from the position where the protrusion 211s is formed. Alternatively, the protrusion 211s may be provided to extend along the circumference of a curved surface intersecting with the long axis Le of the lumen passability checking device 201C.

### Third Embodiment

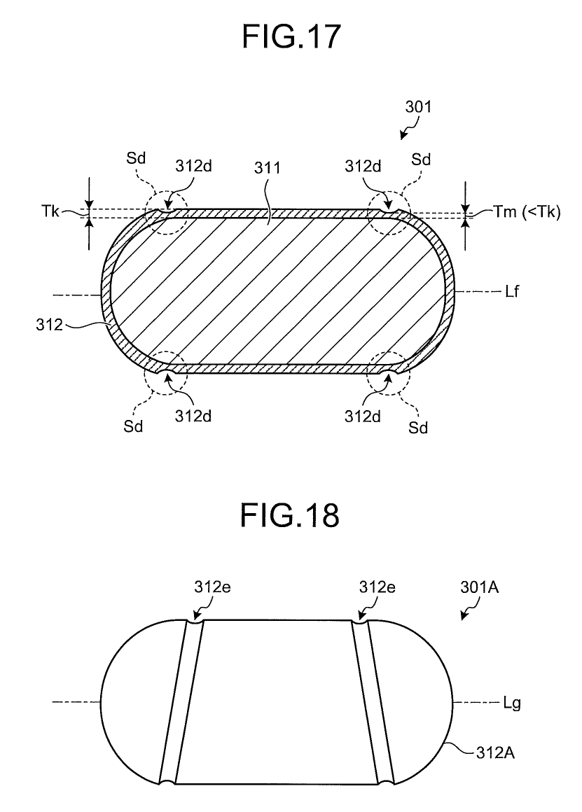

Next, a third embodiment will be described. FIG. 16 is a side view of a lumen passability checking device according to a third embodiment. FIG. 17 is a cross-sectional view of a lumen passability checking device 301 according to the third embodiment, cut along a plane that includes a long axis Lf of the lumen passability checking device 301 and that is parallel to the long axis Lf.

As illustrated in FIGS. 16 and 17, the lumen passability checking device 301 is configured such that a main body 311 has a capsule shape with a smooth surface, and a coating layer 312 encloses the main body 311. On the surface of the coating layer 312, two recesses 312d are provided to extend along the circumference of the cross section orthogonal to the long axis Lf of the lumen passability checking device 301. The recess 312d is formed in a manner such that the whole surface of the main body 311 is coated with an enteric material, or the like, that forms the coating layer in a uniform thickness, and thereafter, the recess 312d is formed with embossing using a protruding member, or by engraving a groove on the surface of the coating layer.

With this configuration, the coating layer 312 is configured such that a thickness Tm of the recess 312d inside a region Sd is thinner than a thickness Tk of the other portions, as illustrated in FIG. 17. Accordingly, in a case where the lumen passability checking device 301 stagnates at a stenosis portion of the small intestine, the coating layer 312 comes to a state where the thin portion, in thickness, on the recess 312d dissolves prior to the other portions, and a hole is generated to open along the circumference of the cross section orthogonal of the long axis Lf of the lumen passability checking device 301, corresponding to the position where the recess 312d is formed, and then, the coating layer 312 is divided into end portions and the cylindrical portion. With this configuration, the coating layer 312 comes to a state where at least the cylindrical portion has its both ends thereof communicating with each other, and this enables intestinal fluid, or the like, to pass through the internal portion of the coating layer 312, making it possible to have an effect similar to the effects of the first embodiment. Alternatively, the recess 312d may be provided to extend along the circumference of a curved surface intersecting with the long axis Lf of the lumen passability checking device 301.

### First Modification Example of Third Embodiment

FIG. 18 is a side view of a lumen passability checking device according to a first modification example of the third embodiment. As illustrated in FIG. 18, a lumen passability checking device 301A according to the first modification example of the third embodiment is configured such that two recesses 312e that extend along the cross section intersecting with a long axis Lg of the lumen passability checking device 301A are formed on a coating layer 312A. Even in a case of the lumen passability checking device 301A, where the two recesses 312e that extend along the circumference of the cross section intersecting with the long axis Lg of the lumen passability checking device 301A are provided on the surface of the coating layer 312A, it is also possible to divide the coating layer remaining inside the body, into end portions and the cylindrical portion. Alternatively, the recess 312e may be provided to extend along the circumference of a curved surface intersecting with the long axis Lg of the lumen passability checking device 301A.

### Second Modification Example of Third Embodiment

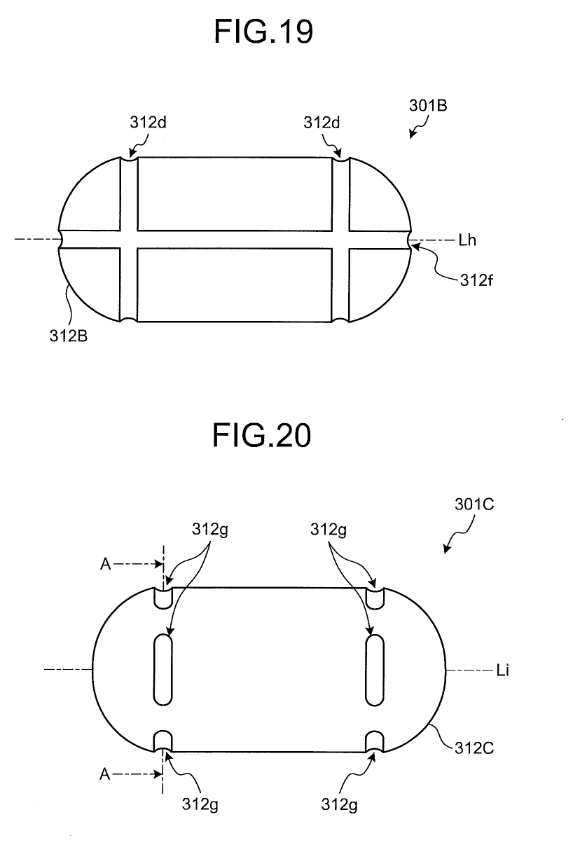

FIG. 19 is a side view of a lumen passability checking device according to a second modification example of the third embodiment. As illustrated in FIG. 19, a lumen passability checking device 301B according to the second modification example of the third embodiment is configured such that a recess 312f is further formed on the surface of a coating layer 312B. The recess 312f is provided to extend along the circumference of the cross section cut at a plane including a long axis Lh of the lumen passability checking device 301B. With this configuration, it is possible to further divide the coating layer 312B of the lumen passability checking device 301B, along the plane passing through the long axis Lh, compared with the lumen passability checking device 301.

### Third Modification Example of Third Embodiment

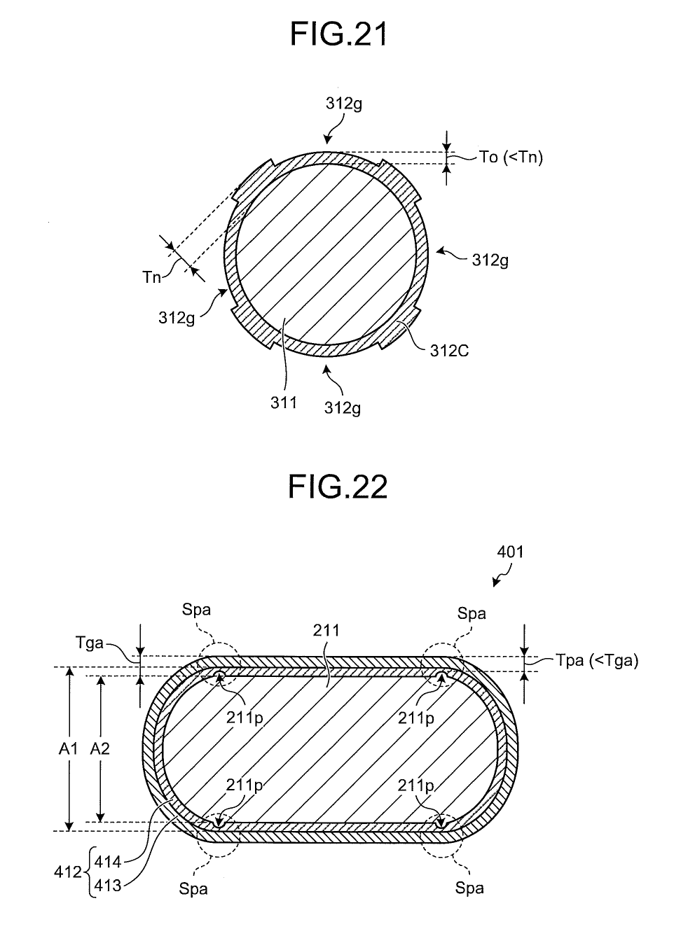

FIG. 20 is a side view of a lumen passability checking device according to a third modification example of the third embodiment. FIG. 21 is a cross-sectional view of FIG. 20, taken along line A-A.

As illustrated in FIGS. 20 and 21, a lumen passability checking device 301C according to the third modification example of the third embodiment is configured such that a coating layer 312C includes two sets of a plurality of recesses 312g partially formed along the circumference of the cross section orthogonal to a long axis Li of the lumen passability checking device 301C. Also in this case, on the coating layer 312C, a thickness To of the portion including the recess 312g is thinner than a thickness Tn of the other portions. In a case where the lumen passability checking device 301C stagnates at a stenosis portion of the small intestine, the coating layer 312C comes to a state where the recess 312g, that is thin in thickness, dissolves prior to the other portions, and perforation-like holes are generated to open along the position of the recess 312g. With this arrangement, the coating layer is easily divided into both end portions and the cylindrical portion, from the position including the recess 312g. Alternatively, the recess 312g may be provided to extend along the circumference of a curved surface intersecting with the long axis Li of the lumen passability checking device 301C.

### Fourth Embodiment

Next, a fourth embodiment will be described. The fourth embodiment relates to a lumen passability checking device capable of checking lumen passability for different-sized capsule endoscopes.

FIG. 22 is a cross-sectional view of the lumen passability checking device according to the fourth embodiment, cut along a plane that includes a long axis of the lumen passability checking device and that is parallel to the long axis. As illustrated in FIG. 22, a lumen passability checking device 401 according to the fourth embodiment includes a coating layer 412 having two layers, compared with the lumen passability checking device 201 according to the second embodiment. Similarly to the second embodiment, the coating layer 412 is configured such that a thickness Tpa of a portion coating the outer surface of the protrusion 211p of the main body 211, indicated with a region Spa, is thinner than a thickness Tga of a portion coating the other portions of the main body 211, so as to allow the remaining coating layer 412 to be divided into end portions and the cylindrical portion in a case where the lumen passability checking device 401 stagnates at a stenosis portion of the small intestine.

The coating layer 412 includes a first coating layer 413 as an outer layer and a second coating layer 414 as an inner layer. The second coating layer 414 coats the surface of the main body 211 and the first coating layer 413 coats the second coating layer 414. In order to check the lumen passability of a capsule endoscope having an outer diameter A1, the first coating layer 413 has an inner diameter A1 equal to the outer diameter A1 of the capsule endoscope. In order to check the lumen passability of a capsule endoscope having an outer diameter A2 (<outer diameter A1), the second coating layer 414 has an inner diameter A2 equal to the outer diameter A2 of the capsule endoscope. That is, in the fourth embodiment, the outer diameter of the main body 211 is equal to the outer diameter A2 of the capsule endoscope.

The first coating layer 413 dissolves in the small intestine and the large intestine, specifically, dissolves in the small intestine and the large intestine in about 40 hours, for example. The second coating layer 414 dissolves in the small intestine and the large intestine, specifically, dissolves in the small intestine and the large intestine in about 30 hours, for example. The thickness of each of the first coating layer 413 and the second coating layer 414 is adjusted such that each of the layers dissolves in a desired time in the small intestine and the large intestine.

The first coating layer 413 and the second coating layer 414 contain different discriminating materials. The first coating layer 413 and the second coating layer 414 include X-ray contrast agents with different concentrations, as the discriminating materials. The first coating layer 413 includes an X-ray contrast agent with concentration D1. The second coating layer 414 includes an X-ray contrast agent with concentration D2. The concentration D2 of the X-ray contrast agent included in the second coating layer 414 is set to be higher than the concentration D1 of the X-ray contrast agent included in the first coating layer 413, so as to be able to differentiate the boundary between the first coating layer 413 and the second coating layer 414 when X-ray imaging is performed.

The lumen passability checking examination using the lumen passability checking device 401 will be described. First, the lumen passability checking device 401 is orally ingested by the subject K, and patency check is performed in about 30 minutes after ingestion, for example. In a case where the lumen passability checking device 401 has been discharged from the body, it is possible to determine that there is patency for the capsule endoscope with the outer diameter A1. In addition, when the patency in the small intestine region with a small lumen size has been confirmed, it would possible to consider that patency for the whole gastrointestinal tract has been confirmed. Therefore, in a case where the lumen passability checking device 401 has reached the large intestine, namely, the organ following the small intestine, and when the first coating layer 413 remains on the whole surface of the device, it is possible to determine that there is patency for the capsule endoscope with the outer diameter Al. In this case, an X-ray image of the lumen passability checking device 401 inside the body of the subject K is captured, and a remaining state of the first coating layer 413 is determined on the basis of a result of imaging.

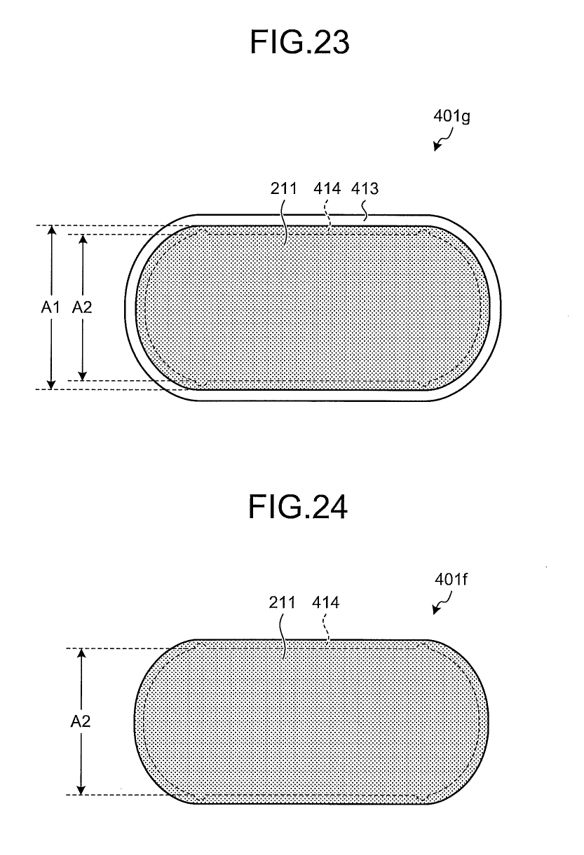

FIG. 23 is a diagram illustrating an X-ray photographic image of the lumen passability checking device 401 in a state where the first coating layer 413 remains on the whole surface of the device. Since the concentration D2 of the X-ray contrast agent of the second coating layer 414 as an inner layer is higher than the concentration D1 of the X-ray contrast agent of the first coating layer 413 as an outer layer, the second coating layer 414 is imaged thicker, on the X-ray image, than the first coating layer 413, making it possible to recognize the boundary between the first coating layer 413 and the second coating layer 414. Specifically, as illustrated as an image 401g in FIG. 23, in a case where the first coating layer 413 remains on the whole surface of the device, the image of the lumen passability checking device 401 appears such that the main body 211 and the second coating layer 414 are imaged as a thick capsule image, and the first coating layer 413 is imaged in a thin image as a layer surrounding the outer periphery of the capsule image. In a case where the image 401g as illustrated in FIG. 23 is positioned in the large intestine in the X-ray imaging for the lumen passability check, it is possible to grasp that the lumen passability checking device 401 has been able to reach the large intestine with the outermost first coating layer 413 remaining on the whole surface of the device. Accordingly, it is possible to determine that the capsule endoscope with the outer diameter A1 equal to the inner diameter Al of the first coating layer 413 can pass through the small intestine, and thus, to evaluate that there is patency for the capsule endoscope with the outer diameter A1.

Furthermore, if it is difficult to grasp a fact that the lumen passability checking device 401 has been discharged from the body or has reached the large intestine, a patency check is performed again in about 60 hours after the lumen passability checking device 401 was ingested. In this case, since 60 hours have elapsed after ingestion of the lumen passability checking device 401, it is possible to determine that the first coating layer 413 has dissolved inside the intestine. Therefore, in a case where the lumen passability checking device 401 has been discharged from the body at this point, it is possible to determine that there is patency for the capsule endoscope with the outer diameter A2. In another case where the lumen passability checking device 401 has reached the large intestine, and the first coating layer 413 has dissolved and the second coating layer 414 is exposed, it is possible to determine that there is patency for the capsule endoscope with the outer diameter A2. Even in this case, an X-ray image of the lumen passability checking device 401 inside the body of the subject K is captured, and whether the second coating layer 414 is in an exposed state is determined on the basis of a result of imaging.

FIG. 24 is a diagram illustrating an X-ray photographic image of the lumen passability checking device 401 in a state where the first coating layer 413 has dissolved and the second coating layer 414 is exposed. Since 60 hours have already elapsed after ingestion, the outermost first coating layer 413 has dissolved. In a case where the second coating layer 414 inside the first coating layer 413 is exposed, the lumen passability checking device 401 can obtain an image 401f on which the main body 211 and the second coating layer 414 are imaged as a thick capsule image (refer to FIG. 24). In a case where the image 401f is positioned in the large intestine, it is possible to grasp that the lumen passability checking device 401 has been able to reach the large intestine with the second coating layer 414 as an inner layer remaining on substantially the whole surface of the device. Accordingly, it is possible to determine that the capsule endoscope with the outer diameter A2 equal to the inner diameter A2 of the second coating layer 414 can pass through the small intestine, and thus, to evaluate that there is patency for the capsule endoscope with the outer diameter A2. In contrast, in a case where the image in which the second coating layer 414 has not remained on the surface of the device, it is possible to evaluate that there is no patency for the capsule endoscope with the outer diameter A2, for the subject K.

According to the fourth embodiment, it is possible to achieve the similar effects to those of the first embodiment. In addition, the two coating layers are provided and contain X-ray contrast agents with different concentrations to discriminate between the two coating layers on the X-ray image. With this structure, it is possible to evaluate patency for the capsule endoscopes with different sizes (outer diameters A1 and A2) in a single patency evaluation examination.

In the fourth embodiment, in order to distinguish between the first coating layer 413 and the second coating layer 414 on the X-ray image, one or more of concentration of the X-ray contrast agents, particle size of the X-ray contrast agents, and layer thickness may differ between the first coating layer 413 and the second coating layer 414.

The dissolution rate of the first coating layer 413 as an outer layer may be higher than the dissolution rate of the second coating layer 414 as an inner layer. With this configuration, it is possible to extend a period of time for which the second coating layer 414 alone remains in the small intestine or the large intestine after the first coating layer 413 has completely dissolved in the small intestine and the large intestine. Accordingly, even when the small intestine contains a lumen site substantially equivalent in size to the outer diameter A2, it is possible to appropriately perform patency check on the capsule endoscope with the outer diameter A2. In this case, one or more of concentration of the enteric material contained in the first coating layer 413 and the second coating layer 414, particle size of the enteric material, and layer thickness may differ between the first coating layer 413 and the second coating layer 414 such that the dissolution rate of the first coating layer 413 is higher than the resolution rate of the second coating layer 414.

Instead of the X-ray contrast agent as a discriminating material for discriminating the first coating layer 413 from the second coating layer 414, a dye may be employed as a discriminating material. In this case, it would be sufficient to allow the first coating layer 413 and the second coating layer 414 to contain dyes of different colors that can be differentiated visually, and to check the color of the surface of the lumen passability checking device discharged from the body. In a case where the color of the surface of the lumen passability checking device that has been discharged from the body is the color that corresponds to the dye contained in the first coating layer 413, it is possible to determine that the lumen passability checking device has been discharged from the body with the first coating layer 413 remaining on the whole surface of the device. Accordingly, it is possible to determine that there is patency for the capsule endoscope with the outer diameter A1. In a case where the color of the surface of the lumen passability checking device that has been discharged from the body is the color that corresponds to the dye contained in the second coating layer 414, it is possible to determine that the lumen passability checking device has been discharged from the body with the first coating layer 413 having dissolved and with the second coating layer 414 remaining on the whole surface of the device. Accordingly, it is possible to determine that there is patency for the capsule endoscope with the outer diameter A2. In a case where the color of the surface of the lumen passability checking device that has been discharged from the body is not any of the color that corresponds to the dye contained in the first coating layer 413 and the color that corresponds to the dye contained in the second coating layer 414, it is determined that both the first coating layer 413 and the second coating layer 414 have dissolved and do not remain on the surface. Accordingly, it is possible to evaluate that there is no patency even for the capsule endoscope with the outer diameter A2, for the subject K.

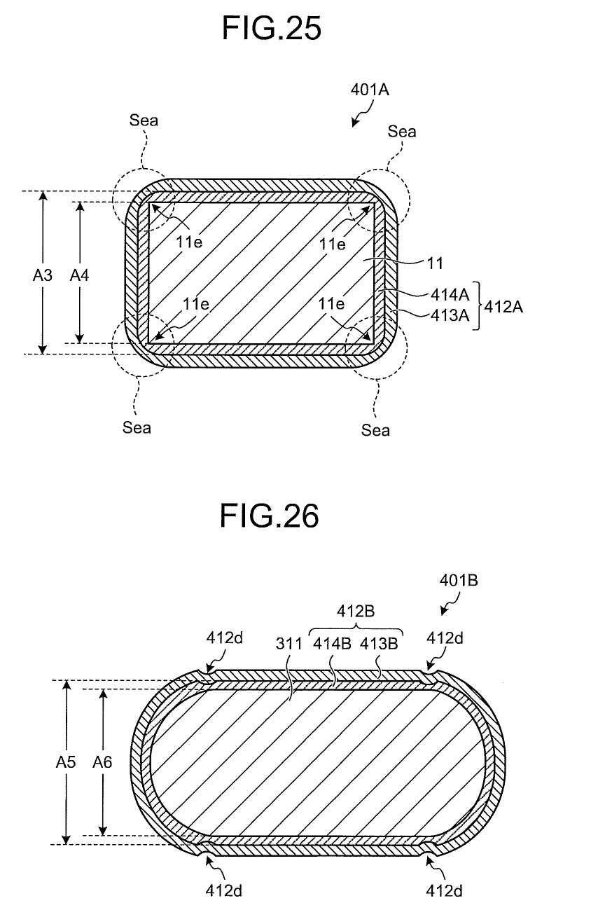

It would be also possible to apply the fourth embodiment to the first embodiment. FIG. 25 is a cross-sectional view of another lumen passability checking device according to the fourth embodiment, cut along a plane that includes a long axis of the lumen passability checking device and that is parallel to the long axis. The coating layer 12 of the lumen passability checking device 1 according to the first embodiment may be configured to include a coating layer 412A having two layers of a first coating layer 413A and a second coating layer 414A containing different discriminating materials to provide a lumen passability checking device 401A in FIG. 25, so as to allow patency check on the capsule endoscope having the outer diameter equal to the inner diameter A3 of the first coating layer 413A and on the capsule endoscope having an outer diameter equal to the inner diameter A4 of the second coating layer 414A. Of course, similarly to the first embodiment, in the coating layer 412A, a portion coating the outer surface of the circumference portion 11e of the main body 11 is thinner than the portion coating the other portions of the main body 11, as illustrated in a region Sea.

It would be also possible to apply the fourth embodiment to the third embodiment. FIG. 26 is a cross-sectional view of another lumen passability checking device according to the fourth embodiment, cut along a plane that includes a long axis of the lumen passability checking device and that is parallel to the long axis. The coating layer 312 of the lumen passability checking device 301 according to the third embodiment may be configured to includes coating layer 412B having two layers of a first coating layer 413B and a second coating layer 414B containing different discriminating materials to provide a lumen passability checking device 401B in FIG. 26, so as to allow patency check on the capsule endoscope having the outer diameter equal to the inner diameter A5 of the first coating layer 413B and on the capsule endoscope having an outer diameter equal to the inner diameter A6 of the second coating layer 414B. Of course, the coating layer 412B includes two recesses 412d, similarly to the third embodiment.

### Fifth Embodiment

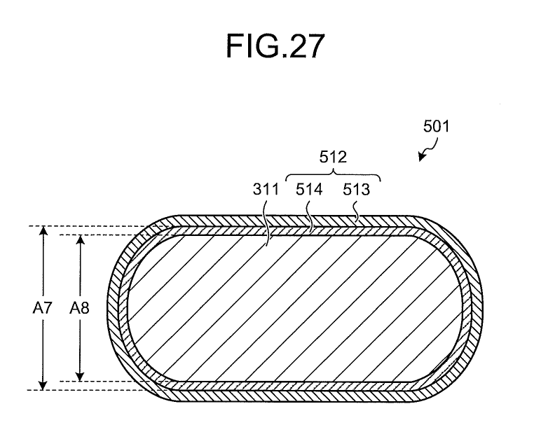

Next, a fifth embodiment will be described. FIG. 27 is a cross-sectional view of the lumen passability checking device according to the fifth embodiment, cut along a plane that includes a long axis of the lumen passability checking device and that is parallel to the long axis.

As illustrated in FIG. 27, a lumen passability checking device 501 according to the fifth embodiment is configured such that a coating layer 512 is formed on the surface of the main body 311. The thickness of the coating layer 512 is substantially equivalent on any of the portions. The coating layer 512 includes a first coating layer 513 and a second coating layer 514 The first coating layer 513 and the second coating layer 514 include different discriminating materials, similarly to the fourth embodiment.

Accordingly, the lumen passability checking device 501 can perform patency check on the capsule endoscope having an outer diameter equal to the inner diameter A7 of the first coating layer 513 and on the capsule endoscope having an outer diameter equal to the inner diameter A8 of the second coating layer 514, and thus can evaluate patency on the capsule endoscopes with different sizes (outer diameters A7 and A8) in a single patency evaluation examination.

Of course, similarly to the fourth embodiment, even in the fifth embodiment, the dissolution rate of the first coating layer 513 as an outer layer may be higher than the dissolution rate of the second coating layer 514 as an inner layer.

Furthermore, according the first to fifth embodiments and the modification examples of the present invention, it is checked whether a capsule medical device is allowed to pass through a small intestine as a gastrointestinal tract, and the lumen passability checking device reaches a large intestine. However, the capsule medical device may reach any organ in gastrointestinal tract between esophagus and the large intestine as long as the organ corresponds to the gastrointestinal tract as a passability check target. In this case, a coating layer containing a discriminating material for checking whether the lumen passability checking device has reached the organ where the capsule medical device is expected to reach, may be configured by using a material dissolvable by a substance existing in the gastrointestinal tract as a passability check target, or existing in the organ where the capsule medical device is expected to reach.

A lumen passability checking device according to some embodiments includes a main body made of a material dissolvable by a substance existing in the gastrointestinal tract, a dissolving layer coating a surface of the main body, having an outer diameter substantially equivalent to an outer diameter in a minor axis of a capsule medical device and being made of a material dissolvable by a substance existing in the gastrointestinal tract. The dissolving layer includes at least two patterns extending along a circumference of a cross section intersecting with a long axis of the lumen passability checking device. With a configuration in which at least a part of the pattern of the dissolving layer is thinner than the other parts of the dissolving layer so as to be dissolved quickly, the dissolving layer can be divided at a desired portion. With this configuration, the dissolving layer would not remain for a long time with a capsule shape even when the dissolving layer remains inside the body of the subject.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A lumen passability checking device for checking whether a capsule medical device configured to be introduced into a gastrointestinal tract of a subject is allowed to pass through the gastrointestinal tract, the device comprising:
a main body made of a material dissolvable by a substance existing in the gastrointestinal tract;
a first dissolving layer coating a surface of the main body, having an outer diameter substantially equivalent to an outer diameter in a minor axis of the capsule medical device, and being made of a material dissolvable by the substance existing in the gastrointestinal tract; and
a second dissolving layer coating a surface of the first dissolving layer and being made of a material dissolvable by the substance existing in the gastrointestinal tract, wherein
the first and second dissolving layers contain different discriminating materials, and
the first dissolving layer has at least two regions extending along a circumference of a cross section of the first dissolving layer, the cross section intersecting with a long axis of the lumen passability checking device, at least a part of each of the two regions being thinner than other parts.

2. The lumen passability checking device according to claim 1, wherein
the main body is columnar,
the first dissolving layer has a first portion coating a boundary between a bottom surface and a side surface of the columnar main body, and has a second portion coating the bottom surface and the side surface of the columnar main body, and
the first portion is thinner than the second portion.

3. The lumen passability checking device according to claim 1, wherein
the main body has one or more protrusions on the surface thereof along a circumference of a cross section of the main body, the cross section intersecting with the long axis of the lumen passability checking device,
the first dissolving layer has a first portion coating the one or more protrusions of the main body and has a second portion coating other parts of the main body, and
the first portion is thinner than the second portion.

4. The lumen passability checking device according to claim 1, wherein
each of the first and second dissolving layers has one or more recesses on each surface thereof along a circumference of a cross section of each of the first and second dissolving layers, the cross section intersecting with the long axis of the lumen passability checking device.

5. The lumen passability checking device according to claim 1, wherein
the discriminating materials are contrast agents.

6. The lumen passability checking device according to claim 5, wherein
one of layer thickness, concentration of the contrast agents, and particle size of the contrast agents differs between the first and second dissolving layers .

7. The lumen passability checking device according to claim 1, wherein
the discriminating materials are dyes.

8. The lumen passability checking device according to claim 1, wherein
a dissolution rate of the second dissolving layer is higher than a dissolution rate of the first dissolving layer.

9. The lumen passability checking device according to claim 1, wherein
a dissolution rate of the main body is higher than a dissolution rate of each of the first and second dissolving layers.

10. The lumen passability checking device according to claim 1, wherein
the second dissolving layer has an inner diameter substantially equivalent to an outer diameter of one of two capsule medical devices, the two capsule medical devices having different outer diameters in minor axes thereof and being configured to be introduced into the subject, and
the first dissolving layer has an inner diameter substantially equivalent to an outer diameter of the other one of the two capsule medical devices.
